(19) United States
(12) Patent Application Publication
(10) Pub. No.: US 2019/0134408 A1
(43) Pub. Date: May 9, 2019

(54) CYCLIC HEATING AND COOLING OF SKIN TO POWER THERMIONIC IMPLANT

(71) Applicant: QUALCOMM Incorporated, San Diego, CA (US)

(72) Inventors: William Henry VON NOVAK, III, San Diego, CA (US); Cody WHEELAND, San Diego, CA (US)

(21) Appl. No.: 16/162,154

(22) Filed: Oct. 16, 2018

Related U.S. Application Data
(60) Provisional application No. 62/582,392, filed on Nov. 7, 2017.

(51) Int. Cl. A61N 1/378; H01L 35/32

(52) U.S. Cl. CPC A61N 1/3787; A61N 1/0504; H01L 35/32

(57) ABSTRACT

Charging a medical implant is performed through the use of an external thermionic implant charging unit placed on the skin of the patient, proximate to the location where a subcutaneous thermionic implant is located. A voltage input can cause the thermionic implant charging unit to enter a warming or cooling phase in which the voltage creates a temperature gradient across a thermoelectric heat pump of the charging unit, causing the charging unit to respectively warm or cool the patient's skin. The thermionic implant can similarly have a thermoelectric heat pump, and the warming or cooling of the skin caused by the charging unit can create a temperature gradient across the thermoelectric heat pump of the thermionic implant, causing its thermoelectric heat pump to create a voltage. This voltage can be used to charge a battery of (and/or directly power) the thermionic implant.

### RELATED APPLICATIONS

This application claims the benefit of U.S. Provisional Application No. 62/582,392, filed Nov. 7, 2017, entitled “CYCLIC HEATING AND COOLING OF SKIN TO POWER THERMIONIC IMPLANT”, which is assigned to the assignee hereof, and incorporated by reference herein in its entirety.

### BACKGROUND

### 1. Field

The subject matter disclosed herein relates generally to subcutaneous electronic medical implants and, more particularly, systems and methods enabling thermal charging of the medical implants.

### 2. Information

Electronic medical implants comprise components, such as sensors and/or stimulators, powered by electricity. Accordingly, such medical implants will typically include a battery as a power source. However, the lifetime of the medical implant may exceed the lifetime of the battery. Extracting the implant (e.g., via surgery) to replace the battery would cause extreme discomfort and risk to the patient in which the medical implant is implanted, so wireless means of charging medical implant batteries have traditionally been used.

Traditional techniques of wireless charging, however, have their shortcomings. These techniques typically utilize an external charging device creating electromagnetic field from which the medical implant can draw power using, for example, a coiled antenna. But there are limits to the amount exposure a patient's body can have to such an electromagnetic field. Furthermore, because the electromagnetic frequency provided by the external charging device must be generated at the frequency to which the antenna of the medical implant must is tuned, the patient may be incapable of recharging the medical implant in scenarios in which the external charging device is not working properly or the patient does not have access to the external charging device.

### SUMMARY

Techniques described herein address these and other issues by providing for the charging a medical implant through the use of an external thermionic implant charging unit placed on the skin of the patient, proximate to the location where a subcutaneous thermionic implant is located. A voltage input can cause the thermionic implant charging unit to enter a warming or cooling phase in which the voltage creates a temperature gradient across a thermoelectric heat pump of the charging unit, causing the charging unit to respectively warm or cool the patient's skin. The thermionic implant can similarly have a thermoelectric heat pump, and the warming or cooling of the skin caused by the charging unit can create a temperature gradient across the thermoelectric heat pump of the thermionic implant, causing its thermoelectric heat pump to create a voltage. This voltage can be used to charge a battery of (and/or directly power) the thermionic implant.

An example thermionic implant charging unit, according to the description comprises a heat exchanger component, and a thermoelectric heat pump having a first surface coupled to the heat exchanger component and a second surface, opposite the first surface, configured to be coupled with skin of a patient. The thermoelectric heat pump comprises an electrical input configured to receive a first voltage causing the thermionic implant charging unit to enter a cooling phase in which the first voltage causes a temperature of the second surface of the thermoelectric heat pump to be cooler than a temperature of the first surface of the thermoelectric heat pump, and receive a second voltage causing the thermionic implant charging unit to enter a warming phase in which the second voltage causes the temperature of the second surface of the thermoelectric heat pump to be warmer than the temperature of the first surface of the thermoelectric heat pump.

Alternative embodiments of the thermionic implant charging unit may comprise one or more of the following features. The thermionic implant charging unit may comprise control circuitry configured to provide voltages to the electrical input of the thermoelectric heat pump, the voltages comprising the first voltage and the second voltage. The control circuitry may be further configured to adjust a duration of the cooling phase, a duration of the warming phase, or both, based on a user input. The control circuitry may be further configured to adjust an amplitude of the first voltage, an amplitude of the second voltage, or both, based on a user input. The control circuitry may be further configured to provide a pulse width modulated (PWM) signal when transitioning from the cooling phase to the warming phase, from the warming phase to the cooling phase, or both. The control circuitry may be further configured to turn the thermoelectric heat pump off for a period of time when transitioning from the cooling phase to the warming phase, when transitioning from the warming phase to the cooling phase, or both. The heat exchanger may comprise a heatsink. The second surface may be configured to be coupled with the skin of the patient via a thermally-conductive member coupled to the second surface of the thermoelectric heat pump.

A method of operating a thermionic implant charging unit, according to the description, comprises providing a first voltage to a thermoelectric heat pump of the thermionic implant charging unit, wherein the thermionic implant charging unit has a first surface coupled to a heat exchanger component and a second surface, opposite the first surface, coupled with skin of a patient, and the first voltage causes the thermionic implant charging unit to enter a cooling phase by causing a temperature of the second surface of the thermoelectric heat pump to be cooler than a temperature of the first surface of the thermoelectric heat pump. The method further comprises providing a second voltage to the thermoelectric heat pump, wherein the second voltage causes the thermionic implant charging unit to enter a warming phase by causing the temperature of the second surface of the thermoelectric heat pump to be warmer than the temperature of the first surface of the thermoelectric heat pump.

Alternative embodiments of the method may comprise one or more of the following features. The method may further comprise receiving a user input. The method may further comprise adjusting a duration of the cooling phase, a duration of the warming phase, or both, based on the user input. The method may further comprise adjusting an amplitude of the first voltage, an amplitude of the second voltage, or both, based on the user input. The method may further comprise providing a pulse width modulated (PWM) signal when transitioning from the cooling phase to the warming phase, from the warming phase to the cooling phase, or both. The method may further comprise turning off the thermoelectric heat pump for a period of time when transitioning from the cooling phase to the warming phase, when transitioning from the warming phase to the cooling phase, or both. The heat exchanger may comprise a heatsink. The second surface may be coupled with the skin of the patient via a thermal conductor coupled to the second surface of the thermoelectric heat pump. The thermal conductor may comprise a thermally-conductive member.

An example device for charging a thermionic implant, according to the description, comprises means for providing a first voltage to a thermoelectric heat pumping means of the device, wherein the device has a first surface coupled to a heat exchange means and a second surface, opposite the first surface, configured to be coupled with skin of a patient, and the first voltage causes the device to enter a cooling phase by causing a temperature of the second surface of the thermoelectric heat pumping means to be cooler than a temperature of the first surface of the thermoelectric heat pumping means. The device further comprises means for providing a second voltage to the thermoelectric heat pumping means, wherein the second voltage causes the device to enter a warming phase by causing the temperature of the second surface of the thermoelectric heat pumping means to be warmer than the temperature of the first surface of the thermoelectric heat pumping means.

Alternative embodiments of the device of claim may comprise one or more of the following features. The device may comprise means for receiving a user input. The device may comprise means for adjusting a duration of the cooling phase, a duration of the warming phase, or both, based on the user input. The device may comprise means for adjusting an amplitude of the first voltage, an amplitude of the second voltage, or both, based on the user input. The device may comprise means for providing a pulse width modulated (PWM) signal when transitioning from the cooling phase to the warming phase, from the warming phase to the cooling phase, or both. The device may comprise means for turning off the thermoelectric heat pump for a period of time when transitioning from the cooling phase to the warming phase, when transitioning from the warming phase to the cooling phase, or both. The heat exchange means may comprise a heatsink. The second surface may be configured to be coupled with the skin of the patient via a thermally conducting means coupled to the second surface of the thermoelectric heat pumping means.

An example non-transitory computer-readable medium, according to the description, has instructions embedded thereon for operating a thermionic implant charging unit. The instructions, when executed by one or more processing units, cause the one or more processing units to provide a first voltage to a thermoelectric heat pump of the thermionic implant charging unit, wherein the thermionic implant charging unit has a first surface coupled to a heat exchanger component and a second surface, opposite the first surface, coupled with skin of a patient, and the first voltage causes the thermionic implant charging unit to enter a cooling phase by causing a temperature of the second surface of the thermoelectric heat pump to be cooler than a temperature of the first surface of the thermoelectric heat pump. The instructions, when executed by one or more processing units, further cause the one or more processing units to provide a second voltage to the thermoelectric heat pump, wherein the second voltage causes the thermionic implant charging unit to enter a warming phase by causing the temperature of the second surface of the thermoelectric heat pump to be warmer than the temperature of the first surface of the thermoelectric heat pump.

Alternative embodiments of the non-transitory computer-readable medium may include one or more of the following features. The instructions, when executed by the one or more processing units, may further cause the one or more processing units to adjust a duration of the cooling phase, a duration of the warming phase, or both, based on a user input. The instructions, when executed by the one or more processing units, may further cause the one or more processing units to adjust an amplitude of the first voltage, an amplitude of the second voltage, or both, based on a user input. The instructions, when executed by the one or more processing units, may further cause the one or more processing units to provide a pulse width modulated (PWM) signal when transitioning from the cooling phase to the warming phase, from the warming phase to the cooling phase, or both.

### BRIEF DESCRIPTION OF DRAWINGS

Non-limiting and non-exhaustive aspects are described with reference to the following figures, wherein like reference numerals refer to like parts throughout the various figures unless otherwise specified.

FIG. 1 is a simplified cross-sectional diagram illustrating an embodiment of a thermionic implant charging system.

FIG. 2 is a simplified block diagram of the electrical components of the thermionic implant charging system illustrated in FIG. 1.

FIGS. 3A-3C are illustrations of waveforms, showing an electrical output (in volts) of the control circuitry over time, according to some embodiments.

FIG. 4A illustrates a pulse width modulated (PWM) waveform used for transitioning from a warming phase to a cooling phase, according to an embodiment.

FIG. 4B illustrates another transitional waveform, according to an embodiment

FIG. 5 is a flow diagram of a method of operating a thermionic implant charging unit, according to an embodiment.

FIG. 6 is a flow diagram of a method of operating a thermionic implant, according to an embodiment.

### DETAILED DESCRIPTION

Thermionic devices (also known as Peltier devices, Peltier heat pumps, solid-state refrigerators, thermoelectric heat pumps, or thermoelectric coolers) are capable of thermoelectric conversion, creating electricity from a temperature gradient, and vice versa. Thermionic devices have been used to power small personal devices such as watches by using a thermal gradient created between a person's body and the surrounding air to generate power (on the order of milliwatts) to power the personal device directly and/or charge a battery of the personal device. Thermoelectric heat pumps have generally not been used to charge devices implanted inside the human body because the relative uniformity of internal body temperatures prevents the creation of a temperature gradient. But charging a medical implant in this manner can offer advantages over traditional wireless radio frequency (RF) implant charging described above.

Techniques described herein are directed toward charging a medical implant through the use of an external thermionic implant charging unit placed on the skin of the patient, proximate to the location where a subcutaneous thermionic implant is located. A voltage input can cause the thermionic implant charging unit to enter a warming or cooling phase in which the voltage creates a temperature gradient across a thermoelectric heat pump of the charging unit, causing the charging unit to respectively warm or cool the patient's skin. The thermionic implant can similarly have a thermoelectric heat pump, and the warming or cooling of the skin caused by the charging unit can create a temperature gradient across the thermoelectric heat pump of the thermionic implant, causing its thermoelectric heat pump to create a voltage. This voltage can be used to charge a battery of (and/or directly power) the thermionic implant.

As described herein, the charging unit can undergo various warming and cooling phases during a charging session to help increase efficiency of the charging by ensuring a temperature gradient across the thermoelectric heat pump of the thermionic implant is maintained. These phases may be adjusted, at least to a degree, based on user input and/or other factors, and the duration, duty cycle, and/or intensity (amplitude) of the phases may be controlled by a control unit. The drives the phases of the thermoelectric heat pump of the charging unit.

Several illustrative embodiments will now be described with respect to the accompanying drawings, which form a part hereof. The ensuing description provides embodiment(s) only, and is not intended to limit the scope, applicability or configuration of the disclosure. Rather, the ensuing description of the embodiment(s) will provide those skilled in the art with an enabling description for implementing an embodiment. It is understood that various changes may be made in the function and arrangement of elements without departing from the spirit and scope of this disclosure.

It will be understood by a person of ordinary skill in the art that, although the embodiments provided herein are directed toward medical applications, the techniques described herein may be utilized in other applications involving the thermal powering and/or charging of electronic devices. Additionally, embodiments provided herein describe the use of a “medical implant,” although such implants may be utilized to gather data and/or stimulate a body part without necessarily performing a medical function. Also, it will be understood that, although various features are described as being “thermionic,” other means of thermoelectric conversion may be used that are not necessarily based on thermionic emission. A person of ordinary skill in the art will recognize many variations.

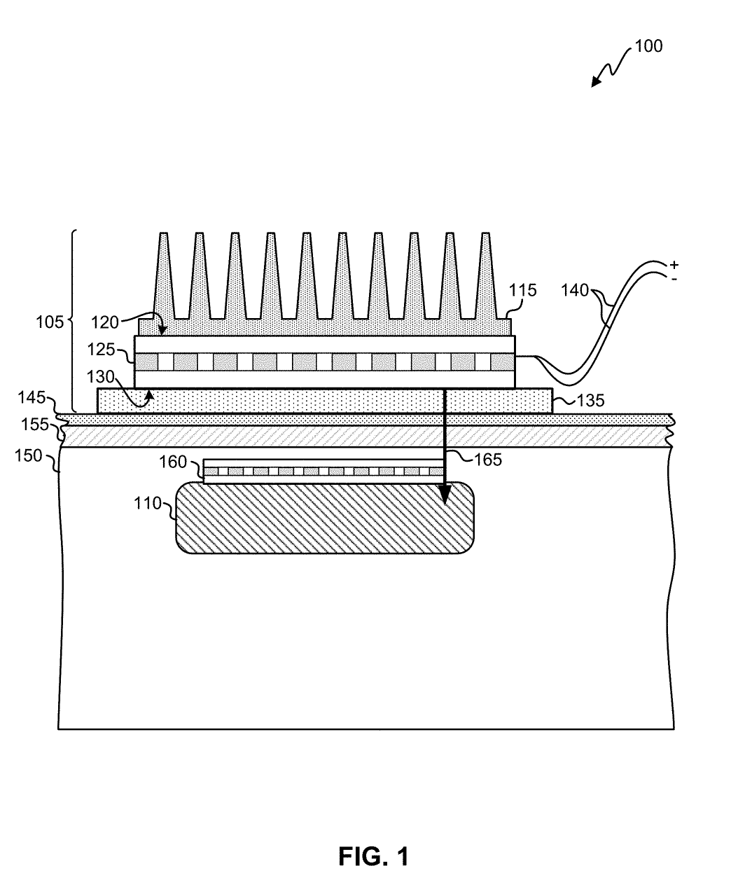

FIG. 1 is a simplified cross-sectional diagram illustrating an embodiment of a thermionic implant charging system 100. Here, a thermionic implant charging unit 105 is used to charge a thermionic implant 110 located within the body of a patient. As with other figures provided herein, FIG. 1 is provided as a non-limiting example. A person of ordinary skill in the art will appreciate that, embodiments are not so limited. Alternative embodiments may include variations in which one or more components may be combined, separated, omitted, added, and/or rearranged, according to desired functionality.

The thermionic implant charging unit 105 comprises a heat exchanger component 115 coupled to a first surface 120 of a thermoelectric heat pump 125. A second surface 130 of the thermoelectric heat pump 125 may be coupled to a thermally-conductive member 135. In some embodiments, the thermionic implant charging unit 105 may comprise a thermally-conductive member 135 permanently coupled to the second surface 130 of the thermoelectric heat pump 125. In other embodiments, the thermally-conductive member 135 may be temporarily coupled to the second surface 130 of the thermoelectric heat pump 125 during use, thereby enabling the use of a removable thermally-conductive member 135 that may be disposable or reusable.

The type of heat exchanger component 115 utilized by the thermionic implant charging unit 105 can vary, depending on factors such as cost, desired thermal dissipation, etc. As illustrated in FIG. 1, some embodiments may include a heat exchanger component 115 comprising a passive heatsink, such as an aluminum or ceramic heatsink with a fin structure. Other embodiments may include a heat exchanger component 115 comprising an active heatsink and/or other types of active or passive thermal exchange/dispersion. In some embodiments, the heat exchanger component 115 may comprise a material similar to the thermally conductive member 135 (discussed in more detail below), which can provide thermal conductivity and/or flexibility for comfort of the patient.

The thermoelectric heat pump 125 comprises a thermionic element, Peltier device, or other thermoelectric conversion device that receives a voltage at an electrical input 140, causing the thermoelectric heat pump 125 to create a thermal gradient between the first surface 120 and the second surface 130. The polarity of the voltage provided at the electrical input 140 (typically a DC voltage; e.g., 12 V) can determine which surface is relatively cool and which surface is relatively warm, and the amplitude of the voltage can determine the temperature difference between the two surfaces. For instance, a relatively small positive voltage may cause the first surface 120 to be slightly cooler than the second surface 130, but a relatively large negative voltage may cause the first surface 120 to be much warmer than the second surface 130. Adjusting the input voltage can control how the thermionic implant charging unit 105 charges the thermionic implant 110. This input voltage can be provided by a control unit (not shown), which is discussed in more detail below.

A thermally-conductive member 135 comprises a member that allows for thermal conduction between the thermoelectric heat pump 125 and the skin 145 of a user. In some embodiments, the thermally-conductive member 135 may comprise a flexible material that may adapt to the shape of the user skin 145, providing more comfort during the charging process. In some embodiments, for example, this material may comprise silicone or a silicone-based product, such as Sil-Pad®. As previously mentioned, in some embodiments, the thermally-conductive member 135 may be permanently coupled to the second surface 130 of the thermoelectric heat pump 125. In other embodiments, the thermally-conductive member 135 may removably coupled to the second surface 130 during the charging process, via an adhesive, physical fasterner, and/or applied pressure, for example. In some embodiments, the thermally-conductive member 135 may comprise a disposable or reusable sticker. In some embodiments, the thermally-conductive member 135 may be incorporated into clothing and/or other wearable items that do not need to be removed during the charging process. In some embodiments, an alternative type of thermal conductor, such as a thermally conductive gel or paste, may be used in addition or as an alternative to the thermally-conductive member 135.

During the charging process, a temperature gradient generated by the thermoelectric heat pump 125 creates a temperature on the second surface 130 different than the temperature of a volume 150 internal to the body of a user into which the thermionic implant 110 is implanted. This creates a temperature gradient in a direction illustrated by arrow 165, across the thermally-conductive member 135, skin 145, and subcutaneous fat 155 of the user, and across a thermoelectric heat pump receiver 160 of the thermionic implant 110. (Here, the thermionic implant 110 can act as a heatsink for the thermoelectric heat pump receiver 160. As such, materials may be chosen for the body of the thermionic implant 110 to facilitate thermal conduction.) The thermal electric heat pump receiver 160 of the thermionic implant 110 can then utilize this temperature gradient to generate power for the thermionic implant 110 (providing direct power and/or charging a battery of the thermionic implant 110).

The thermionic implant 110 itself may comprise any of a variety of devices and may be located in any of a variety of subcutaneous locations within the body of the patient. It can be noted that, although FIG. 1 illustrates the thermionic implant 110 is illustrated as being located under both the skin 145 and the subcutaneous fat 155, the thermionic implant 110 may be located at any of a variety of depths underneath the skin. 145, including in or above the subcutaneous fat 155. As discussed in more detail below, the depth of the thermionic implant 110 may impact how it is charged, where the durations of heating and cooling phases generally increase with an increase of the depth of the thermionic implant 110.

The location of the thermionic implant 110 may also depend on its functionality. The thermionic implant 110 may comprise any of a variety of devices including, for example, blood sugar monitors, vital signs monitors (e.g., pulse monitors), etc. To this end, the thermionic implant 110 may include any of a variety of electrical circuitry and other components, including various types of sensors, and/or stimulators. Such sensors and/or stimulators may need to work at different locations within the body, and at different depths underneath the skin.

The thermionic implant charging system 100 provides a relatively “low-tech” implant charging solution, which has its advantages. In addition to avoiding electromagnetic field exposure to the body and the need to accommodate relatively inefficient antennas in the design of an implant, the thermionic implant charging system 100 can allow a user to charge the thermionic implant 110 using relatively low-tech means. For example, in instances where the thermionic implant charging unit 105 is unavailable or nonfunctional, a user may utilize different means for heating and/or cooling the skin 145 (e.g., hot/cold water, heating/cooling pads, etc.) to charge the thermionic implant 110, thereby avoiding potential failure of the thermionic implant 110.

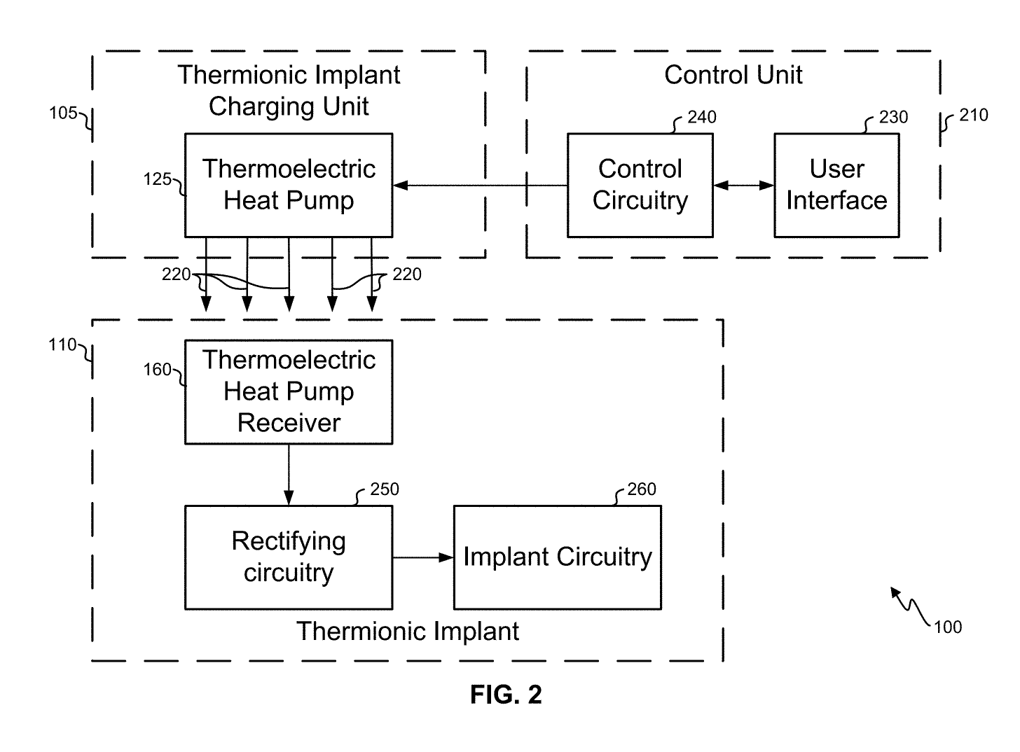

FIG. 2 is a simplified block diagram of the electrical components of the thermionic implant charging system 100 illustrated in FIG. 1. The block diagram of the thermionic implant charging system 100 of FIG. 2 is provided as a supplement to FIG. 1 to help further illustrate functionality. Arrows 220 illustrate thermal coupling. Other arrows illustrate electrical (data and/or power) connections. FIG. 2 illustrates, in the addition to the components illustrated in FIG. 1, a control unit 210 and various other components not explicitly illustrated in FIG. 1. A person of ordinary skill in the art will appreciate that not all electrical components are represented in FIG. 2 (e.g., power sources) and that other components (e.g., communication interfaces) may be included, depending on desired functionality. In alternative embodiments, various components can be combined, separated, or otherwise altered, depending on desired functionality. For example, in some embodiments the control unit 210 may be wholly or partially integrated into the thermionic implant charging unit 105.

According to techniques herein, a patient or healthcare provider can charge the thermionic implant 110, at home, a hospital, clinic, etc. The frequency of the charging may depend on factors such as battery capacity and power usage of the thermionic implant 110. The capacity of the battery (not shown) of the thermionic implant 110 may be selected to balance size concerns (e.g., a large battery may not be practical with certain types of implants) with convenience. For example, it may be inconvenient for patient to have to recharge the thermionic implant 110 on a daily basis in certain applications. However, recharging the thermionic implant 110 on a weekly or longer basis may be considered acceptable in these applications.

Prior to the charging process, a user (e.g., the patient or a healthcare provider) can place the thermionic implant charging unit 105 on a portion of the patient's skin proximate to the thermionic implant 110. (Depending on the depth at which the thermionic implant 110 is located, the thermionic implant 110 may be visible through the patient's skin. In any case, the user can locate the thermionic implant 110 and place the thermionic implant charging unit 105 on a surface of the skin in proximity to the thermionic implant.) To maximize thermal coupling (illustrated by arrows 220) between the thermoelectric heat pump 125 of the thermionic implant charging unit 105, it may be preferable to position the thermionic implant 110 such that the thermoelectric heat pump receiver 160 is closest to the portion of the scan on which the thermionic implant charging unit 105 will be placed during charging, as illustrated in FIG. 1.

Once the thermionic implant charging unit 105 is in position, the user can initiate the charging process using the control unit 210. For example, the user may power on the control unit 210 and/or provide an input using the user interface 230 of the control unit 210 to cause the control circuitry 240, to provide voltages to the thermoelectric heat pump 125 of the thermionic implant charging unit 105. As previously indicated, the thermionic implant charging unit 105 may enter a warming phase or a cooling phase, depending on the polarity of the voltage input provided by the control circuitry 240, and the thermal coupling between the thermoelectric heat pump 125 and thermoelectric heat pump receiver 160 causes a thermal gradient across the thermoelectric heat pump receiver 160, allowing it to output a voltage to the rectifying circuitry 250, which then provides a rectified output voltage to the implant circuitry 260.

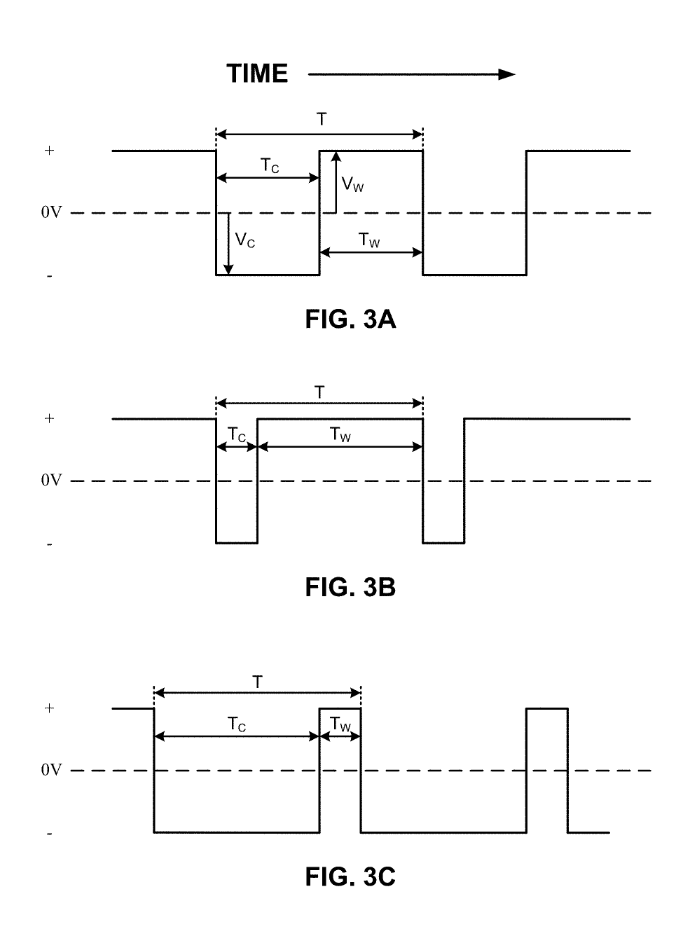

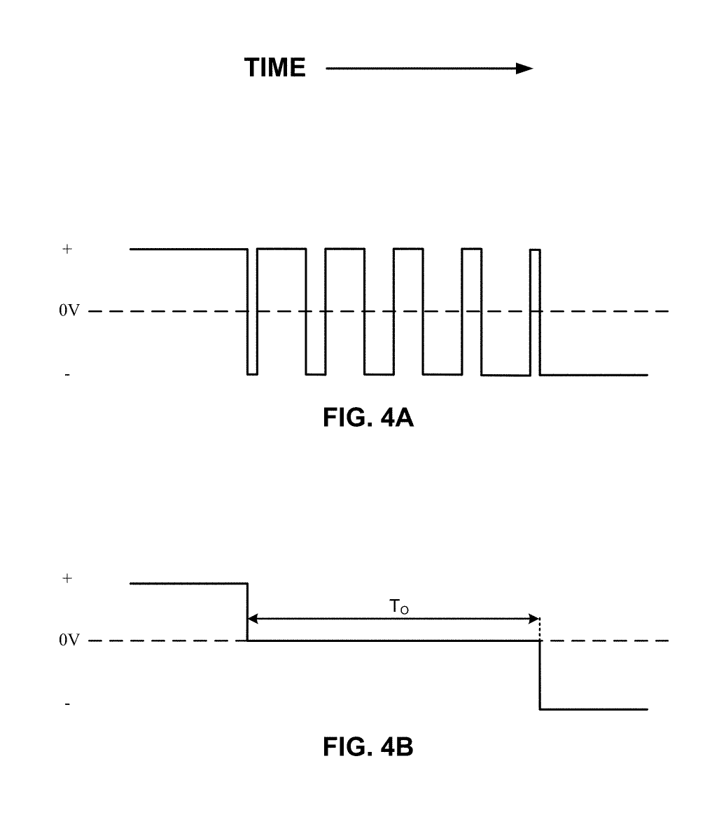

One way to help ensure that a thermal gradient is maintained across the thermoelectric heat pump receiver 160 of the thermionic implant 110 is to cycle between warming and cooling phases. FIGS. 3A-3C and 4A-4B illustrate how this can be done, according to some embodiments.

FIGS. 3A-3C are illustrations of waveforms, showing an electrical output (in volts) of the control circuitry 240 over time, according to some embodiments. Here, a negative voltage, V_{C}, output by the control unit 210 causes the thermionic implant charging unit 105 to enter a cooling phase for a certain period of time (known as a “dwell time”) T_{C}. A positive voltage, V_{W}, causes the thermionic implant charging unit 105 to enter a warming phase for a dwell time T_{W}. A cycle comprising one cooling phase followed by a warming phase lasts a total duration of time T. Cycles may be repeated several times over the course of a charging session.

Cyclic charging in this manner can take advantages of two phenomena to help ensure efficient charging of the thermionic implant 110. First, cyclic charging takes advantage of the fact that the patient's body takes time to heat up and cool down, thereby making it possible to maintain a heating differential. Second, the cyclic process taps into large temperature differentials when switching from a heating cycle to a cooling cycle, and vice versa, again, helping ensure a heating differential is maintained on the thermoelectric heat pump receiver 160 of the thermionic implant 110.

The dwell times T_{W} and T_{C} can vary, depending on desired various factors. If dwell times T_{W} and T_{C} are too short, there may not be sufficient time to create a temperature differential on the thermoelectric heat pump receiver 160. And if they are too long, temperature dissipation within the body of the patient may reach a steady-state in which the temperature differential on the thermoelectric heat pump receiver 160 is reduced. Either situation could lead to inefficiencies. Factors such as implant depth, skin thickness, tissue type, and the like may impact what the optimal periods of time might be for dwell times T_{W} and T_{C}. As such, a doctor or other healthcare provider may be able to configure the control unit 210 to provide maximum and minimum full-time for T_{W} and/or T_{C}, to help ensure efficiency and charging in view of the factors of the particular thermionic implant 110. In general, dwell times T_{W} and T_{C} may typically range from roughly 10 seconds to several minutes, although certain applications may have longer or shorter dwell times.

Similarly, the amplitudes V_{W} and V_{C} may also vary, depending on desired factors. Generally speaking, the greater the amplitude, the greater temperatures produced by the thermoelectric heat pump 125. In other words, the greater the amplitude of V_{W}, the warmer the thermostatic heat pump 125 will cause the skin to be during a warming phase; and likewise, the greater the amplitude of V_{C}, the cooler the thermostatic heat pump 125 will cause the skin to be during a cooling phase. Thus, the amplitudes V_{W} and V_{C} may be set to ensure the comfort of the patient (ensuring temperatures are within, for example, 50° F. to 130° F.). In embodiments where amplitudes V_{W} and V_{C} may be adjusted by a user, maximum amplitudes may be set (e.g., by the manufacturer, healthcare provider, and/or patient) to ensure patient comfort. Minimum amplitudes may also be set to help ensure efficiency during the charging session.

In some embodiments, a user interface 230 may include a touchscreen, dial, or other input mechanism to allow adjustment of one or both of the amplitudes V_{W} and V_{C} and/or one or both of the dwell times T_{W} and T_{C}. FIGS. 3B-3C illustrate how user may be able to adjust dwell times T_{W} and T_{C} to accommodate comfort of the patient and/or other concerns. Here, depending on desired functionality, the cycle duration T may be the same and the duty cycle may shift based on user input. Other embodiments may additionally or alternatively allow for the adjustment of the cycle duration.

If, for example, a patient prefers a warmer charging session, the patient may be able to provide an input (e.g., using the user interface 230 of the control unit 210) to adjust the duty cycle of the waveform such that the dwell time T_{W} of the warming phase is longer than the dwell time T_{C} of the cooling phase, as illustrated in FIG. 3B, resulting in more time during the charging session spent in the warming phase than the cooling phase. If the patient prefers a cooler charging session, the patient can similarly provide input to adjust the duty cycle of the waveform such that the dwell time T_{W} of the warming phase is shorter than the dwell time T_{C} of the cooling phase, as illustrated in FIG. 3C, resulting in more time during the charging session spent in the cooling phase than the warming phase. Depending on desired functionality, the granularity of these adjustments can be course (e.g., three settings: “warm,” “cold,” and “neutral”) or fine (e.g., having a large number of settings between a “warm” extreme and a “cold” extreme).

Factors in addition or alternative to patient comfort may be considered when adjusting the duty cycle of dwell times T_{W} and T_{C}. Ambient temperature may be a factor. If it is relatively hot, for example, a longer cooling cycle may make the charging session more efficient. A medical prescription may also impact dwell times T_{W} and T_{C}. For instance, heating and/or cooling times may be established for pain relief of the area. Depth and location of the implant can also impact duty cycle. (It can be noted that some or all of these factors may additionally or alternatively be considered for adjustments to amplitude.)

Heat dissipation takes time, and therefore the temperatures felt by the patient at or below the skin 145 will change much more slowly than the waveforms of FIGS. 3A-3C. (As such, the temperatures felt by the patient will not have the sharp edges of these waveforms.) Nevertheless, factors such as amplitudes V_{W} and V_{C}, efficiency of the thermoelectric heat pump 125, thermal conductivity of the thermally-conductive member 135, and the like may cause relatively quick changes in temperatures between heating and cooling phases, which may cause discomfort to the patient.

Depending on desired functionality, additional waveforms may be utilized to address patient comfort and/or other concerns. FIGS. 4A and 4B illustrate to additional waveforms that can be used to cause a slower transition between warming and cooling phases.

FIG. 4A illustrates a pulse width modulated (PWM) waveform used for transitioning from a warming phase to a cooling phase, according to an embodiment. Here, PWM is used to smooth the transition from a heating phase to a cooling phase.

FIG. 4B illustrates another transitional waveform, according to an embodiment. Here, the control circuitry 240 provide 0V to the thermoelectric heat pump 125, thereby turning the thermoelectric heat pump 125 off for a period of time T_{O}. This allows the skin 145 to cool off naturally for a period of time after warming phase before beginning the cooling phase.

The duration of the waveforms illustrated in FIGS. 4A-4B may be static or may be adjustable (e.g., within a range). Such adjustments may be made by a user via the user interface 230, for example. Again, because sharper changes in temperature result in higher temperature gradients and more power generated by the thermoelectric heat pump receiver 160 of the thermionic implant 110, a manufacturer or healthcare provider may limit the adjustability of these waveforms to help ensure the charging session maintains a threshold level of efficiency

Returning again to FIG. 2, the thermionic implant 110 can be configured to provide power to the implant circuitry 260 (e.g. charging a battery, and/or providing direct power to the implant circuitry 260) during both heating and cooling phases. That is, the rectifying circuitry 250 can be configured to provide a positive output voltage regardless of whether the input voltage from the output of the thermoelectric heat pump receiver is positive or negative.

Components within the rectifying circuitry 250 can vary, depending on desired functionality. As indicated above, heating and cooling phases are relatively slow, lasting 10 seconds or more. Thus, the components of the rectifying circuitry 250 may be selected accordingly. Because the size of the thermionic implant 110 and thermoelectric heat pump receiver 160 may be relatively small, the resulting temperature differential across the thermoelectric heat pump receiver 160 may also be relatively small, resulting in a correspondingly small DC output to the rectifying circuitry 250 (e.g., roughly 0.5 V to 1 V). In some embodiments, to deal with these relatively low voltages, a synchronous rectifier may be implemented using switches such as field effect transistors (FETs) rather than diodes. The implant circuitry 260 may use a boost converter to get the voltage up to a useful range. A person of ordinary skill in the art will appreciate various other considerations.

Depending on the functionality of the implant circuitry 260, the thermionic implant 110 may be able to provide data to the control unit via, for example, wireless radio frequency (RF) communication. In some embodiments, for example, implant circuitry 260 may comprise a wireless communication interface enabling the thermionic implant 110 to communicate wirelessly (e.g., using near field communication (NFC), Bluetooth® low energy (BLE), and/or another low-power wireless technology). As such, the implant circuitry 260 may provide data to the control unit via wireless communications, allowing the control unit to receive some feedback during a charging session. This feedback, which can include a charge status of the battery, output voltage of the thermoelectric heat pump receiver 160 and/or rectifying circuitry 250, and the like, can enable the control unit 210 to ensure efficient charging process. This can be ensured by, for example, adjusting amplitudes and/or dwell times of voltage waveforms provided to the thermoelectric heat pump 125 to help maximize an output voltage of the thermoelectric heat pump receiver 160 and/or rectifying circuitry 250, stopping the charging session once a battery is charged, and/or calculating on-the-fly how long a charging session could last and how adjusting a duty cycle may affect this. Additionally or alternatively, the thermionic implant 110 may wirelessly communicate some or all of this data to a mobile device (e.g., a user's cell phone), which can determine this information (charge time, amplitude and/or duty cycle adjustments, etc.) and provide this information to a user, who may make amplitude and/or duty cycle adjustments using the user interface 230 of the control unit 210 based on this information.

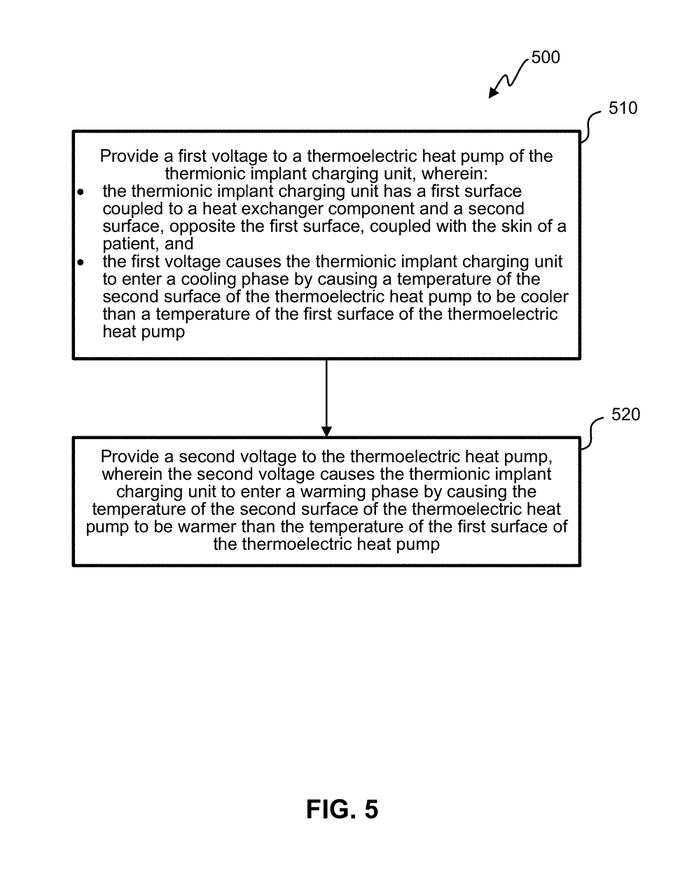

FIG. 5 is a flow diagram 500 of a method of operating a thermionic implant charging unit, according to an embodiment. As with other figures provided herein, FIG. 5 is provided as a non-limiting example. Alternative embodiments may include additional functionality to that shown in the figure, and/or the functionality shown in one or more of the blocks in the figure may be omitted, combined, separated, and/or performed simultaneously. Means for performing the functionality of the blocks may include one or more hardware and/or software components of a control unit 210, such as control circuitry 240, which may comprise a microprocessor, integrated circuit, and/or other circuitry configured to perform the method of FIG. 5.

A person of ordinary skill in the art will recognize many variations. For example, although described as “first” and “second” voltages, the ordering of these voltages (and the order of the functionality described in the blocks of FIG. 5) may vary. That is, in some instances, the ordering of the voltages shown in flow diagram 500 may be such that the second voltage is initially applied, and the first voltage is applied afterward.

The functionality at block 510 comprises providing a first voltage to a thermoelectric heat pump of the thermionic implant charging unit having a first surface coupled to a heat exchanger component and a second surface, opposite the first surface, coupled to a thermally-conductive member placed on the skin of a patient, wherein the first voltage causes the thermionic implant charging unit to enter a cooling phase by causing a temperature of the second surface of the thermoelectric heat pump to be cooler than a temperature of the first surface of the thermoelectric heat pump.

The functionality of block 520 comprises providing a second voltage to the thermoelectric heat pump, wherein the second voltage causes the thermionic implant charging unit to enter a warming phase by causing the temperature of the second surface of the thermoelectric heat pump to be warmer than the temperature of the first surface of the thermoelectric heat pump.

Some embodiments may include one or more of the following functions. As indicated in the embodiments described above, for example, the first and second voltages may be provided to an electrical input of the thermoelectric heat pump by control circuitry configured to provide voltages to the thermoelectric heat pump. The control circuitry may be further configured to adjust a duration of the cooling phase, a duration of the warming phase, or both, based on user input. The control circuitry may further be configured to adjust an amplitude of the first voltage, an amplitude of the second voltage, or both, based on a user input. In some embodiments, the control circuitry may be further configured to provide a PWM signal when transitioning from the cooling phase to the warming phase, from the warming phase to the cooling phase, or both. Additionally or alternatively, the control circuitry may be configured to turn the thermoelectric heat pump off for a period of time when transitioning from the cooling phase to the warming phase, from the warming phase to the cooling phase, or both. According to some embodiments, the heat exchanger may comprise a heatsink.

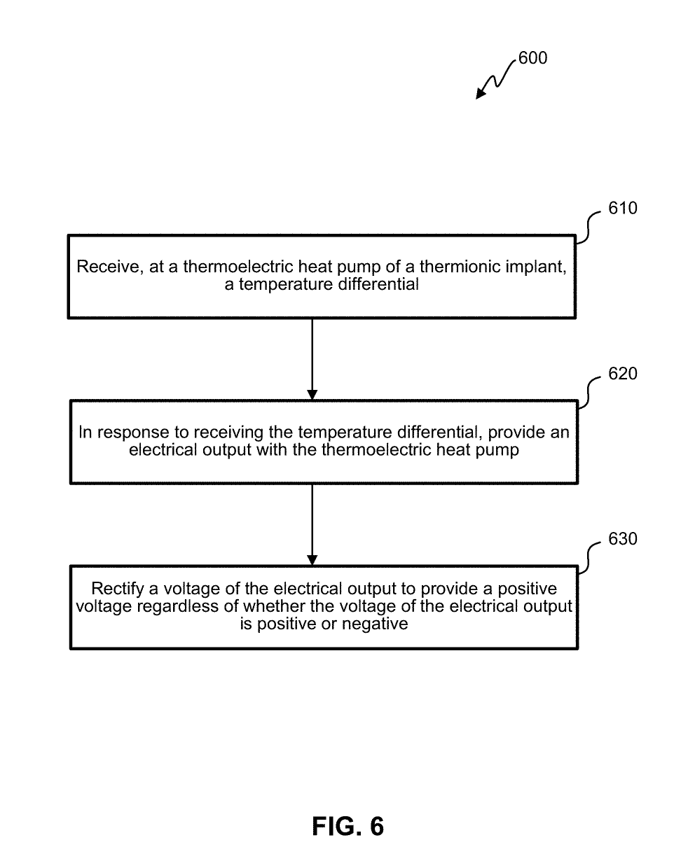

FIG. 6 is a flow diagram 600 of a method of operating a thermionic implant, according to an embodiment. Again, FIG. 6 is provided as a non-limiting example. Alternative embodiments may include additional functionality to that shown in the figure, and/or the functionality shown in one or more of the blocks in the figure may be omitted, combined, separated, and/or performed simultaneously. Means for performing the functionality of the blocks may include one or more hardware and/or software components of a thermionic implant 110, such as a thermoelectric heat pump receiver 160 and/or rectifying circuitry 250. A person of ordinary skill in the art will recognize many variations.

At block 610, the functionality includes receiving, at a thermoelectric heat pump of the thermionic implant, a temperature differential. As indicated in the embodiments described above, the temperature differential may be established during a warming or cooling phase of a charging session in which a thermionic implant charging unit is used to charge the thermionic implant.

At block 620, in response to receiving the temperature differential, an electrical output is provided with the thermoelectric heat pump. Here, the output may have a relatively low voltage in view of the relatively small dimensions of the thermoelectric heat pump.

At block 630, the functionality includes rectifying a voltage of the electrical output to provide a positive voltage regardless of whether the voltage of the electrical output is positive or negative. Such functionality can enable the thermionic implant to be charged during both heating and cooling phases of a charging session, providing better efficiencies and faster charging of the thermionic implant.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

Also, configurations may be described as a process which is depicted as a schematic flowchart or block diagram. Although each may describe the operations as a sequential process, many of the operations can be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

The methods, systems, and devices discussed herein are examples. Various embodiments may omit, substitute, or add various procedures or components as appropriate. For instance, features described with respect to certain embodiments may be combined in various other embodiments. Different aspects and elements of the embodiments may be combined in a similar manner. The various components of the figures provided herein can be embodied in hardware and/or software. Also, technology evolves and, thus, many of the elements are examples that do not limit the scope of the disclosure to those specific examples.

It has proven convenient at times, principally for reasons of common usage, to refer to such signals as bits, information, values, elements, symbols, characters, variables, terms, numbers, numerals, or the like. It should be understood, however, that all of these or similar terms are to be associated with appropriate physical quantities and are merely convenient labels. Unless specifically stated otherwise, as is apparent from the discussion above, it is appreciated that throughout this specification discussions utilizing terms such as “processing,” “computing,” “calculating,” “determining,” “ascertaining,” “identifying,” “associating,” “measuring,” “performing,” or the like refer to actions or processes of a specific apparatus, such as a special purpose computer or a similar special purpose electronic computing device. In the context of this specification, therefore, a special purpose computer or a similar special purpose electronic computing device is capable of manipulating or transforming signals, typically represented as physical electronic, electrical, or magnetic quantities within memories, registers, or other information storage devices, transmission devices, or display devices of the special purpose computer or similar special purpose electronic computing device.

Terms, “and” and “or” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AA, AAB, AABBCCC, etc.

Having described several embodiments, various modifications, alternative constructions, and equivalents may be used without departing from the spirit of the disclosure. For example, the above elements may merely be a component of a larger system, wherein other rules may take precedence over or otherwise modify the application of the invention. Also, a number of steps may be undertaken before, during, or after the above elements are considered. Accordingly, the above description does not limit the scope of the disclosure.

What is claimed is:

1. A thermionic implant charging unit comprising:
a heat exchanger component; and
a thermoelectric heat pump having a first surface coupled to the heat exchanger component and a second surface, opposite the first surface, configured to be coupled with skin of a patient, wherein the thermoelectric heat pump further comprises an electrical input configured to:
receive a first voltage causing the thermionic implant charging unit to enter a cooling phase in which the first voltage causes a temperature of the second surface of the thermoelectric heat pump to be cooler than a temperature of the first surface of the thermoelectric heat pump, and
receive a second voltage causing the thermionic implant charging unit to enter a warming phase in which the second voltage causes the temperature of the second surface of the thermoelectric heat pump to be warmer than the temperature of the first surface of the thermoelectric heat pump.

2. The thermionic implant charging unit of claim 1, further comprising control circuitry configured to provide voltages to the electrical input of the thermoelectric heat pump, the voltages comprising the first voltage and the second voltage.

3. The thermionic implant charging unit of claim 2, wherein the control circuitry is further configured to adjust a duration of the cooling phase, a duration of the warming phase, or both, based on a user input.

4. The thermionic implant charging unit of claim 2, wherein the control circuitry is further configured to adjust an amplitude of the first voltage, an amplitude of the second voltage, or both, based on a user input.

5. The thermionic implant charging unit of claim 2, wherein the control circuitry is further configured to provide a pulse width modulated (PWM) signal when transitioning from the cooling phase to the warming phase, from the warming phase to the cooling phase, or both.

6. The thermionic implant charging unit of claim 2, wherein the control circuitry is further configured to turn the thermoelectric heat pump off for a period of time when transitioning from the cooling phase to the warming phase, when transitioning from the warming phase to the cooling phase, or both.

7. The thermionic implant charging unit of claim 1, wherein the heat exchanger comprises a heatsink.

8. The thermionic implant charging unit of claim 1, wherein the second surface is configured to be coupled with the skin of the patient via a thermally-conductive member coupled to the second surface of the thermoelectric heat pump.

9. A method of operating a thermionic implant charging unit, the method comprising:
providing a first voltage to a thermoelectric heat pump of the thermionic implant charging unit, wherein:
the thermionic implant charging unit has a first surface coupled to a heat exchanger component and a second surface, opposite the first surface, coupled with skin of a patient, and
the first voltage causes the thermionic implant charging unit to enter a cooling phase by causing a temperature of the second surface of the thermoelectric heat pump to be cooler than a temperature of the first surface of the thermoelectric heat pump; and
providing a second voltage to the thermoelectric heat pump, wherein the second voltage causes the thermionic implant charging unit to enter a warming phase by causing the temperature of the second surface of the thermoelectric heat pump to be warmer than the temperature of the first surface of the thermoelectric heat pump.

10. The method of claim 9, further comprising receiving a user input.

11. The method of claim 10, further comprising adjusting a duration of the cooling phase, a duration of the warming phase, or both, based on the user input.

12. The method of claim 10, further comprising adjusting an amplitude of the first voltage, an amplitude of the second voltage, or both, based on the user input.

13. The method of claim 9, further comprising providing a pulse width modulated (PWM) signal when transitioning from the cooling phase to the warming phase, from the warming phase to the cooling phase, or both.

14. The method of claim 9, further comprising turning off the thermoelectric heat pump for a period of time when transitioning from the cooling phase to the warming phase, when transitioning from the warming phase to the cooling phase, or both.

15. The method of claim 9, wherein the heat exchanger comprises a heatsink.

16. The method of claim 9, wherein the second surface is coupled with the skin of the patient via a thermal conductor coupled to the second surface of the thermoelectric heat pump.

17. The method of claim 9, wherein the thermal conductor comprises a thermally-conductive member.

18. A device for charging a thermionic implant, the device comprising:
means for providing a first voltage to a thermoelectric heat pumping means of the device, wherein:
the device has a first surface coupled to a heat exchange means and a second surface, opposite the first surface, configured to be coupled with skin of a patient, and
the first voltage causes the device to enter a cooling phase by causing a temperature of the second surface of the thermoelectric heat pumping means to be cooler than a temperature of the first surface of the thermoelectric heat pumping means; and
means for providing a second voltage to the thermoelectric heat pumping means, wherein the second voltage causes the device to enter a warming phase by causing the temperature of the second surface of the thermoelectric heat pumping means to be warmer than the temperature of the first surface of the thermoelectric heat pumping means.

19. The device of claim 18, further comprising means for receiving a user input.

20. The device of claim 19, further comprising means for adjusting a duration of the cooling phase, a duration of the warming phase, or both, based on the user input.

21. The device of claim 19, further comprising means for adjusting an amplitude of the first voltage, an amplitude of the second voltage, or both, based on the user input.

22. The device of claim 18, further comprising means for providing a pulse width modulated (PWM) signal when transitioning from the cooling phase to the warming phase, from the warming phase to the cooling phase, or both.

23. The device of claim 18, further comprising means for turning off the thermoelectric heat pump for a period of time when transitioning from the cooling phase to the warming phase, when transitioning from the warming phase to the cooling phase, or both.

24. The device of claim 18, wherein the heat exchange means comprises a heatsink.

25. The device of claim 18, wherein the second surface is configured to be coupled with the skin of the patient via a thermally conducting means coupled to the second surface of the thermoelectric heat pumping means.

26. A non-transitory computer-readable medium having instructions embedded thereon for operating a thermionic implant charging unit, the instructions, when executed by one or more processing units, cause the one or more processing units to:
provide a first voltage to a thermoelectric heat pump of the thermionic implant charging unit, wherein:
the thermionic implant charging unit has a first surface coupled to a heat exchanger component and a second surface, opposite the first surface, coupled with skin of a patient, and
the first voltage causes the thermionic implant charging unit to enter a cooling phase by causing a temperature of the second surface of the thermoelectric heat pump to be cooler than a temperature of the first surface of the thermoelectric heat pump; and
provide a second voltage to the thermoelectric heat pump, wherein the second voltage causes the thermionic implant charging unit to enter a warming phase by causing the temperature of the second surface of the thermoelectric heat pump to be warmer than the temperature of the first surface of the thermoelectric heat pump.

27. The non-transitory computer-readable medium of claim 26, wherein the instructions, when executed by the one or more processing units, further cause the one or more processing units to adjust a duration of the cooling phase, a duration of the warming phase, or both, based on a user input.

29. The non-transitory computer-readable medium of claim 26, wherein the instructions, when executed by the one or more processing units, further cause the one or more processing units to adjust an amplitude of the first voltage, an amplitude of the second voltage, or both, based on a user input.

30. The non-transitory computer-readable medium of claim 26, wherein the instructions, when executed by the one or more processing units, further cause the one or more processing units to provide a pulse width modulated (PWM) signal when transitioning from the cooling phase to the warming phase, from the warming phase to the cooling phase, or both.
